(19) United States
(12) Patent Application Publication
(10) Pub. No.: US 2024/0000677 A1
(43) Pub. Date: Jan. 4, 2024

(54) TINTED COSMETIC SUNSCREEN COMPOSITION, USE OF A TINTED COSMETIC SUNSCREEN COMPOSITION, AND PROCESS FOR MANUFACTURING A TINTED COSMETIC SUNSCREEN COMPOSITION

(71) Applicant: L'OREAL, Paris (FR)

(72) Inventors: Paula NEVES PITTA, Rio de Janeiro (BR); Tassia HANASHIRO, Rio de Janeiro (BR); Marie BOISDRON, Shanghai (CN)

(21) Appl. No.: 18/274,607

(22) PCT Filed: Jan. 29, 2021
(86) PCT No.: PCT/BR2021/050044

§ 371 Date: Jul. 27, 2023

(51) Int. Cl. A61K 8/29; A61Q 17/04; A61K 8/19; A61K 8/36; A61K 8/55; A61K 8/92; A61K 8/40; A61K 8/41; A61K 8/37; A61K 8/49; A61K 8/35

(52) U.S. Cl. CPC A61K 8/29; A61Q 17/04; A61K 8/19; A61K 8/361; A61K 8/55; A61K 8/922; A61K 8/40; A61K 8/415; A61K 8/37; A61K 8/4966; A61K 8/35; A61K 2800/43

(57) ABSTRACT

The present invention is directed to new tinted cosmetic sunscreen compositions, comprising (a) at least one colored pigment selected from titanium dioxide, iron oxides and mixtures thereof, (b) glyceryl stearate (and) PEG-100 stearate, (c) potassium cetyl phosphate, and (d) at least one UV filter. The present invention is also directed to the use of a tinted cosmetic sunscreen composition, and to a process for manufacturing a tinted cosmetic sunscreen composition.

### FIELD OF THE INVENTION

The present invention is directed to new tinted cosmetic sunscreen compositions, comprising (a) at least one colored pigment selected from titanium dioxide, iron oxides and mixtures thereof, (b) glyceryl stearate (and) PEG-100 stearate, (c) potassium cetyl phosphate, and (d) at least one UV filter. The present invention is also directed to the use of a tinted cosmetic sunscreen composition, and to a process for manufacturing a tinted cosmetic sunscreen composition.

### BACKGROUND OF THE INVENTION

The photoprotection of keratinous materials, including both skin and hair, is considered of great importance in order to protect from sun-damage, sunburn, photo-aging, as well as to decrease the chances of skin cancer development caused by exposure to ultraviolet (“UV”) radiation. There are typically two types of UVA/UVB cosmetic sunscreen compositions used to accomplish photoprotection, namely, inorganic UV filters and organic UV filters.

The degree of UV protection afforded by a cosmetic sunscreen composition is directly related to the amount and type of UV filters contained therein. Particularly, cosmetic sunscreen compositions must provide good protection against the sun rays, a measure of which is the Sun Protection Factor (SPF) value, a desirable balance between UVA and UVB protection, particularly a minimum UVA protection factor, yet have satisfactory sensory perception, such as a smooth but not greasy feel upon application.

Furthermore, it is also desirable to produce a cosmetic sunscreen composition that not only confers photoprotection of keratinous materials, but also provides additional properties. For example, it is desired to produce a tinted cosmetic sunscreen composition with high SPF and UVA/UVB protection, with the capability of tinting the skin to correct imperfections, preferably with high coverage that is capable of suiting different skin tones.

Also, the market for tinted cosmetic sunscreen compositions is growing and becoming more and more important, and consumers are looking for innovation, such as a cosmetic sunscreen composition with makeup properties or appearance after application.

The degree of coverage provided by a tinted cosmetic sunscreen composition, when applied to the skin, is directly related to its technology and the amount of colored pigments present in the composition.

However, the combination of sunscreen and tinting properties with other sensorial benefits such as dry touch, good spreadability, the presence of different raw materials (fillers, emollients), this complex system with high amount of UV filters and pigments, has been difficult to achieve, particularly because the presence of high amounts of colored pigments and UV filters in tinted cosmetic sunscreen compositions results in stability issues in the formulation, especially the migration of the pigments to the surface of the composition, even creating discoloration spots on said surface. These issues are intensified in darker shades of tinted cosmetic sunscreen compositions.

Therefore, a tinted cosmetic sunscreen composition with high SPF and UVA/UVB protection and a high quantity of colored pigments, in order to provide a desired color coverage when applied to the skin, which is stable, does not feel heavy on the consumer's skin and does not present migration and separations of the colored pigments on the surface of the composition.

The inventors of the present application have surprisingly managed to develop a tinted cosmetic sunscreen composition with high SPF and UVA/UVB protection and high amounts of colored pigments, while the composition had good sensorial attributes and stability.

### SUMMARY OF THE INVENTION

The present invention is directed to new tinted cosmetic sunscreen compositions, comprising (a) at least one colored pigment selected from titanium dioxide, iron oxides and mixtures thereof, (b) glyceryl stearate (and) PEG-100 stearate, (c) potassium cetyl phosphate, and (d) at least one UV filter. The present invention is also directed to the use of a tinted cosmetic sunscreen composition, and to a process for manufacturing a tinted cosmetic sunscreen composition.

Other features and advantages of the present invention will be apparent from the following more detailed description of the desirable embodiments which illustrates, by way of example, the principles of the invention.

### BRIEF DESCRIPTION OF THE DRAWINGS

FIG. 1 demonstrates the results of the Stability Test of Example 5, wherein FIG. 1a is related to the results of the state-of-the-art composition of Ex. 2 and FIG. 1b is related to the results of the inventive tinted cosmetic sunscreen composition according to Ex. 3.

### DETAILED DESCRIPTION OF THE INVENTION

In an embodiment, the tinted cosmetic sunscreen composition of the present invention comprises:
(a) from 5.0 to 6.5% by weight, including all ranges and sub-ranges there between, based on the total weight of the composition, of at least one colored pigment selected from titanium dioxide, iron oxides and mixtures thereof;
(b) from 1.7 to 1.9% by weight, including all ranges and sub-ranges there between, based on the total weight of the composition, of glyceryl stearate (and) PEG-100 stearate;
(c) from 1.4 to 1.6% by weight, including all ranges and sub-ranges there between, based on the total weight of the composition, of potassium cetyl phosphate, and
(d) from 15.0 to 25.0% by weight, including all ranges and sub-ranges there between, based on the total weight of the composition, of at least one UV filter.

In a preferred embodiment, the amount of the at least one colored pigment ranges from about 5.0 to about 6.5%, from about 5.2 to about 6.4%, from about 5.5 to about 6.3%, from about 5.7 to about 6.2%, from about 5.8 to about 6.2%, from about 5.9 to about 6.0%, from about 6.0 to about 6.5% by weight, including all ranges and sub-ranges there between, based on the total weight of the composition.

In a preferred embodiment, the amount of glyceryl stearate (and) PEG-100 stearate ranges from about 1.7 to about 1.9%, from about 1.75 to about 1.85%, from about 1.8 to about 1.9%, from about 1.7 to about 1.8% by weight, including all ranges and sub-ranges there between, based on the total weight of the composition.

In a preferred embodiment, the amount of potassium cetyl phosphate ranges from about 1.4 to about 1.6%, from about 1.45 to about 1.55%, from about 1.5 to about 1.6%, from about 1.4 to about 1.5%, from about 1.4 to about 1.55%, from about 1.5 to about 1.55% by weight, including all ranges and sub-ranges there between, based on the total weight of the composition.

In a preferred embodiment, the at least one UV filter is selected from octocrylene, diethylamino hydroxybenzoyl hexyl benzoate, ethylhexyl salicylate, ethylhexyl triazone, butyl methoxydibenzoylmethane, titanium dioxide, and mixtures thereof.

In a preferred embodiment, the amount of the at least one UV filter ranges from about 15.0 to about 25.0%, from about 16.0 to about 24.0%, from about 17.0 to about 23.0%, from about 18.0 to about 22.0%, from about 19.0 to about 21.0%, from about 20.0 to about 20.5%, from about 20.0 to about 22.0%, from about 20.0 to about 25.0%, from about 18.0 to about 21.0%, by weight, including all ranges and sub-ranges there between, based on the total weight of the composition.

In a preferred embodiment, the tinted cosmetic sunscreen composition further comprises Copernicia cerifera (carnauba) wax, ranging from about 1.0 to about 3.0%, from about 1.5 to about 2.5%, from about 1.75 to about 2.25%, to about 1.6 to about 2.1%, from about 1.7 to about 2.0%, from about 2.5 to about 3.0% by weight, including all ranges and sub-ranges there between, based on the total weight of the composition.

In a preferred embodiment, the tinted cosmetic sunscreen composition further comprises cosmetically acceptable ingredients selected from additional sunscreens, active compounds, fatty compounds, fillers, perfume/fragrance, polymers, preserving agents, solvents, additional surfactants, vitamins, silicones and mixtures thereof.

In a preferred embodiment, the tinted cosmetic sunscreen composition of the present invention presents a Sun Protection Factor (SPF) ranging from 40 to 70, preferably from 50 to 60, more preferably about 50. In various embodiments, the tinted cosmetic sunscreen composition of the present invention may present a Sun Protection Factor of 40, 45, 50, 55, 60, 65, and 70.

In an embodiment, the tinted cosmetic sunscreen composition of the present invention may present a Sun Protection Factor of 50.

The tinted cosmetic sunscreen composition of the present invention is in the form of an oil in water (O/W) emulsion.

The tinted cosmetic sunscreen composition of the present invention can be used as a daily product for the skin.

The present invention also relates to the use of said tinted cosmetic sunscreen composition for manufacturing a product for the photoprotection of keratinous materials with high SPF and UVA/UVB protection and with high color coverage.

### Terms

As used herein, the expression “at least” means one or more and thus includes individual components as well as mixtures/combinations.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The terms “comprising,” “having,” and “including” are used in their open, non-limiting sense.

The compositions and methods of the present disclosure can comprise, consist of, or consist essentially of the essential elements and limitations of the disclosure described herein, as well as any additional or optional ingredients, components, or limitations described herein or otherwise useful.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

All percentages, parts and ratios herein are based upon the total weight of the compositions of the present disclosure, unless otherwise indicated.

Other than in the operating examples, or where otherwise indicated, all numbers expressing quantities of ingredients and/or reaction conditions are to be understood as being modified in all instances by the term “about” which can encompass+/−10%, +/−8%, +/−6%, +/−5%, +/−4%, +/−3%, +/−2%, +/−1%, or +/−0.5%.

“Substituted,” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counter-ion. This list of counter-ions, however, is non-limiting.

All components and elements positively set forth in this disclosure can be negatively excluded from the claims. In other words, the compositions of the instant disclosure can be free or essentially free of all components and elements positively recited throughout the instant disclosure.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### Additional UV Filters

In addition to the at least one UV filter selected from octocrylene, diethylamino hydroxybenzoyl hexyl benzoate, ethylhexyl salicylate, ethylhexyl triazone, butyl methoxydibenzoylmethane, titanium dioxide, and mixtures thereof, the tinted cosmetic sunscreen composition of the present invention may further comprise additional UV filters.

According to the invention, the concentration of additional sunscreen/UV filters in the system may be between about 5.0 to about 35.0%, preferably between about 7.0 to about 30.0% and even more preferably between about 10.0 to about 29.0% by weight, based on the total weight of the composition.

Additional non-limiting suitable UV filters of the present invention could be as follows:

### Oil-Soluble Organic Sunscreen Ingredient

The “oil-soluble organic sunscreen ingredient” means any organic compound for screening out UV radiation, which can be fully dissolved in molecular form or miscible in an oil phase or which can be dissolved in colloidal form (for example in micellar form) in an oil fatty phase.

Non-limiting examples of oil-soluble organic sunscreen ingredients useful in the invention include, for example, cinnamic derivatives; anthranilates; salicylic derivatives; dibenzoylmethane derivatives; camphor derivatives; benzophenone derivatives; diphenylacrylate derivatives; triazine derivatives; benzotriazole derivatives; benzalmalonate derivatives, especially those cited in U.S. Pat. No. 5,624,663; benzimidazole derivatives; imidazolines; bis-benzoazolyl derivatives as described in patents EP669323 and U.S. Pat. No. 2,463,264; p-aminobenzoic acid (PABA) derivatives; methylene bis(hydroxyphenylbenzotriazole) derivatives as described in applications U.S. Pat. Nos. 5,237,071, 5,166,355, GB2303549, DE19726184 and EP893119, benzoxazole derivatives as described in patent applications EP0832642, EP1027883, EP1300137 and DE10162844; screening polymers and screening silicones such as those described especially in patent application WO 93/04665; dimers derived from alkyl-styrene such as those described in patent application DE 19855649; 4,4-diarylbutadienes such as those described in patent applications EP0967200, DE19746654, DE19755649, EP-A-1008586, EP1133980 and EP1133981, merocyanine derivatives such as those described in patent applications WO 04/006878, WO 05/058269 and WO 06/032741; and mixtures thereof, the entire contents of the patents and patent applications being incorporated by reference in their entirety.

As examples of other suitable oil-soluble organic sunscreen ingredients, mention may be made of those denoted herein below under their INCI name:

Cinnamic Derivatives:

Examples of suitable cinnamic derivatives include, but are not limited to, ethyl hexyl methoxycinnamate, isopropyl methoxycinnamate, isoamyl methoxycinnamate, DEA methoxycinnamate, diisopropyl methylcinnamate, glyceryl ethylhexanoate dimethoxycinnamate.

Dibenzoylmethane Derivatives:

Examples of suitable dibenzoylmethane derivatives include, but are not limited to, isopropyl dibenzoylmethane.

Salicylic Derivatives:

Examples of suitable salicylic derivatives include, but are not limited to, homosalate, dipropylene glycol salicylate and TEA salicylate.

Beta, Beta-Diphenylacrylate Derivatives:

Examples of suitable beta, beta-diphenylacrylate derivatives include, but are not limited to, etocrylene.

Benzophenone Derivatives:

Examples of suitable benzophenone derivatives include, but are not limited to, benzophenone-1, benzophenone-2, benzophenone-3 or oxybenzone, benzophenone-4, benzophenone-5, benzophenone-6, benzophenone-8, benzophenone-9, benzophenone-12, n-hexyl 2-(4-diethylamino-2-hydroxybenzoyl)benzoate or as a mixture with octyl methoxycinnamate.

Benzylidenecamphor Derivatives:

Examples of suitable benzylidenecamphor derivatives include, but are not limited to, 3-benzylidene camphor manufactured, 4-methylbenzylidene camphor, polyacrylamidomethyl benzylidene camphor manufactured.

Phenylbenzotriazole Derivatives:

Examples of suitable phenylbenzotriazole derivatives include, but are not limited to, drometrizole trisiloxane, methylene bis-benzotriazolyl tetramethylbutylphenol, or in micronized form as an aqueous dispersion.

Triazine Derivatives:

Examples of suitable triazine derivatives include, but are not limited to, bis-ethylhexyloxyphenol methoxyphenyl triazine, diethylhexyl butamido triazone, 2,4,6-tris(dineopentyl 4′-aminobenzalmalonate)-s-triazine, 2,4,6-tris(diisobutyl 4′-aminobenzalmalonate)-s triazine, 2,4-bis(dineopentyl 4′-aminobenzalmalonate)-6-(n-butyl 4′-aminobenzoate)-s-triazine, symmetrical triazine screening agents described in U.S. Pat. No. 6,225,467, patent application WO 2004/085412 (see compounds 6 and 9) or the document “Symmetrical Triazine Derivatives” IP.COM Journal, IP.COM Inc., West Henrietta, NY, US (20 Sep. 2004), especially 2,4,6-tris(biphenyl)-1,3,5-triazines (in particular 2,4,6-tris(biphenyl-4-yl)-1,3,5-triazine and 2,4,6-tris(terphenyl)-1,3,5-triazine, which is included in patent applications WO 06/035000, WO 06/034982, WO 06/034991, WO 06/035007, WO 2006/034992 and WO 2006/034985).

Anthranilic Derivatives:

An example of a suitable anthranilic derivative includes, but is not limited to, methyl anthranilate.

Imidazoline Derivatives:

An example of a suitable imidazoline derivative includes, but is not limited to, ethylhexyl dimethoxybenzylidene dioxoimidazoline propionate.

Benzalmalonate Derivatives:

An example of a suitable benzalmalonate derivative includes, but is not limited to, polyorganosiloxane containing benzalmalonate functions, for instance polysilicone-15.

4,4-Diarylbutadiene Derivatives:

An example of a suitable 4,4-diarylbutadiene derivative includes, but is not limited to, 1-Dicarboxy(2,2′-dimethylpropyl)-4,4-diphenyl-butadiene.

Benzoxazole Derivatives:

An example of suitable benzoxazole derivative includes, but is not limited to, 2,4-bis[5-(1-dimethylpropyl)benzoxazol-2-yl-(4-phenyl)imino]-6-(2-ethylhexyl)imino-1,3,5-triazine, and mixtures thereof.

Preferably, the oil-soluble organic sunscreen ingredient will be chosen from bis-ethylhexyloxyphenol methoxyphenyl triazine.

The oil-soluble organic sunscreen ingredient is preferably present in the composition according to the invention in an amount of from about 3% to about 25% by weight, preferably in an amount of from about 5% to about 20% by weight, and most preferably about 7% to about 18% by weight, based on the total weight of the composition.

### Water-Soluble Organic Sunscreen Ingredient

The “water-soluble organic sunscreen ingredient” means any organic compound for screening out UV radiation, which can be fully dissolved in molecular form or miscible in a liquid aqueous phase or which can be dissolved in colloidal form (for example in micellar form) in a liquid aqueous phase.

Non-limiting examples of water-soluble organic sunscreen ingredients useful in the invention include, for example, terephthalylidene dicamphor sulfonic acid, phenylbenzimidazole sulfonic acid, benzophenone-4, aminobenzoic acid (PABA), 4-Bis(polyethoxy)-para-aminobenzoic acid polyethoxyethyl ester (PEG-25 PABA), camphor benzalkonium methosulfate, methylene bis-benzotriazolyl tetramethylbutylphenol (Bisoctrizole), disodium phenyl dibenzimidazole tetrasulfonate (Bisdisulizole disodium), and tris-biphenyl triazine; their derivatives and corresponding salts; naphthalene bisimide derivatives such as those described in European patent application EP1990372 A2, the entire contents of which is hereby incorporated by reference; and cinnamido amine cationic quaternary salts and derivatives such as those described in U.S. Pat. No. 5,601,811, the entire contents of which is hereby incorporated by reference, and mixtures thereof.

The salts of the compounds that may be used according to the invention are chosen in particular from salts of alkali metals, for example sodium or potassium; salts of alkaline-earth metals, for example calcium, magnesium or strontium; metal salts, for example zinc, aluminum, manganese or copper; salts of ammonium of formula NH4+; quaternary ammonium salts; salts of organic amines, for instance salts of methylamine, dimethylamine, trimethylamine, triethylamine, ethylamine, 2-hydroxyethylamine, bis(2-hydroxyethyl)amine or tris(2-hydroxyethyl)amine; lysine or arginine salts. Salts chosen from sodium, potassium, magnesium, strontium, copper, manganese or zinc salts are preferably used. The sodium salt is preferably used.

Preferably, the water-soluble organic sunscreen ingredient will be chosen from methylene bis-benzotriazolyl tetramethylbutylphenol, and mixtures thereof.

The water-soluble organic sunscreen ingredient is preferably present in the composition according to the invention in an amount of from about 0.1% to about 10% by weight, preferably in an amount of from about 0.5% to about 8% by weight, and most preferably about 1% to about 7% by weight, based on the total weight of the composition.

### Silica-Coated Titanium Dioxide Sunscreen Ingredient

In addition, the tinted cosmetic sunscreen composition of the present invention may comprise a silica-coated titanium dioxide sunscreen ingredient. The “silica-coated titanium dioxide sunscreen ingredient” means spherical beads which are formed by encapsulating titanium dioxide particles in silica.

Non-limiting examples of silica coated titanium dioxide sunscreen ingredients useful in the invention include, for example, titanium dioxide coated with silica, such as name silica (and) titanium dioxide having a composition of silica: titanium dioxide of about 55:45 and having a particle size from about 2 microns to about 7 microns.

The silica-coated titanium dioxide sunscreen ingredient is preferably present in the composition according to the invention in an amount of from about 1.0 to about 5.0% by weight, preferably in an amount of from about 2.0 to about 3.0% by weight, based on the total weight of the composition.

### Additional Ingredients

In addition to the essential components described hereinbefore, the tinted cosmetic sunscreen composition of the present invention may further comprise any usual cosmetically acceptable ingredient, which may be chosen especially from such as additional UV filters, active compounds, fatty compounds, fillers, perfume/fragrance, polymers, preserving agents, solvents, additional surfactants, vitamins, pH adjuster, silicones and mixtures thereof.

The tinted cosmetic sunscreen composition of the present invention comprises water. The water used may be sterile demineralized water and/or deionized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-Saunier, water from EauxBonnes, water from Rochefort, water from Saint Christau, water from Les Fumades, water from Tercis-les-Bains or water from Avene. Water may also comprise reconstituted thermal water, that is to say a water comprising trace elements such as zinc, copper, magnesium, etc., reconstituting the characteristics of a thermal water.

A person skilled in the art will take care to select the optional additional ingredients and/or the amount thereof such that the advantageous properties of the composition according to the invention are not, or are not substantially, adversely affected by the envisaged addition.

Suitable polymers include, but are not limited to, acrylates/C₁₀₋₃₀ alkyl acrylate crosspolymer, styrene/acrylates copolymer, and mixtures thereof.

The composition may also comprise at least one silicon ingredient, which may be dimethicone and caprylyl methicone, among others.

Non-limiting example of preserving agent which can be used in accordance with the invention include phenoxyethanol, disodium EDTA and mixtures thereof.

Additional suitable solvents include, but are not limited to, 012-15 alkyl benzoate, pentylene glycol, caprylyl glycol, and mixtures thereof.

Adequate surfactants may be selected from potassium cetyl phosphate, sodium methyl stearoyl taurate and inulin lauryl carbamate, among others.

In various embodiments, the solvent is present in a concentration from about 10.0 to about 75.0% by weight, or from about 10.0 to about 50.0% by weight, or from about 30.0 to about 70.0% by weight, or from about 35.0 to about 75.0% by weight, or preferably from about 40.0 to about 70.0% by weight, and more preferably from about 40.0 to about 60.0% by weight, including ranges and sub-ranges therebetween, based on the total weight of the combinations and/or compositions of the present disclosure.

Additionally suitable ingredients include, but are not limited to, pH adjusters such as triethanolamine.

The composition may comprise at least one dye/pigment, preferably titanium oxide or iron oxides.

The composition may comprise vitamins, such as ascorbyl glucoside, tocopherol, as mixtures thereof.

Exemplary fat or oil materials include, but are not limited to, isopropyl lauroyl sarcosinate, stearyl alcohol, and mixtures thereof.

The composition may also comprise at least one filler, such as silica.

The additional ingredients may represent from 50.0 to 85.0%, such as from 55.0 to 82.0% or such as from 60.0 to 80.0% by weight of the total weight of the composition.

By way of non-limiting illustration, the invention will now be described with reference to the following examples.

### Process for Manufacturing a Tinted Cosmetic Sunscreen Composition

A non-limiting example regarding a process for manufacturing a tinted cosmetic sunscreen composition according to the present invention could be as follows:
Step (A): Main Vessel—hot oily phase comprising oil raw materials in a temperature ranging from 75° C. to 80° C.;
Step (B): Side Vessel—hot aqueous phase comprising oil raw materials in a temperature ranging from 75° C. to 80° C.;
Step (C): the aqueous phase of step (B) is added to the oily phase of step (A), followed by mixing the mixture;
Step (D): the polymers are added to the mixture obtained in step (C), subsequently followed by a neutralization;
Step (E): the fillers are gradually added to the mixture obtained in step (D) and mixed until homogeneity, at a temperature of below 40° C.

### EMBODIMENTS

An example of a suitable tinted cosmetic sunscreen composition according to the present invention comprises:
from 5.5% to 6.5% of iron oxides;
from 1.7% to 1.85% of glyceryl stearate (and) peg-100 stearate;
from 1.45% to 1.6% of potassium cetyl phosphate;
from 18.0% to 22.0% of ethylhexyl triazone, titanium dioxide, octocrylene, diethylamino hydroxybenzoyl hexyl benzoate, ethylhexyl salicylate and butyl methoxydibenzoylmethane;
from 0.1% to 1.0% of triethanolamine and disodium EDTA;
from 8.0% to 12% of isononyl isononanoate, stearyl alcohol and Copernicia cerifera (carnauba) wax;
from 2.0% to 3.0% of silica and silica silylate;
from 0.1% to 1.0% of fragrance;
from 1.5% to 3.5% of ammonium acryloyldimethyltaurate/vp copolymer, aluminum starch octenylsuccinate and acrylates/C10-30 alkyl acrylate crosspolymer;
from 0.5% to 1.5% of phenoxyethanol and chlorphenesin;
from 0.5% to 3.0% of glycerin and caprylyl glycol;
from 45.0% to 55.0% of water;
from 0.3% to 3.0% of sodium methyl stearoyl taurate, poloxamer 338 and stearic acid;
from 0.05% to 0.2% of a Citrus limon (lemon) fruit extract; and
from 0.01% to 0.2% of ascorbyl glucoside and tocopherol.

Another suitable tinted cosmetic sunscreen composition according to the present invention comprises:
from 5.5% to 6.5% of iron oxides and titanium dioxide;
from 1.7% to 1.85% of glyceryl stearate (and) peg-100 stearate;
from 1.45% to 1.6% of potassium cetyl phosphate;
from 18.0% to 22% of ethylhexyl triazone, titanium dioxide, octocrylene, diethylamino hydroxybenzoyl hexyl benzoate, ethylhexyl salicylate and butyl methoxydibenzoylmethane;
from 0.1% to 1.0% of triethanolamine and disodium EDTA;
from 8.0% to 12% of isononyl isononanoate, stearyl alcohol and Copernicia cerifera (carnauba) wax;
from 2.0% to 3.0% of silica and silica silylate;
from 0.1% to 1.0% of fragrance;
from 1.5% to 3.5% of ammonium acryloyldimethyltaurate/vp copolymer, aluminum starch octenylsuccinate and acrylates/C10-30 alkyl acrylate crosspolymer;
from 0.5% to 1.5% of phenoxyethanol and chlorphenesin;
from 0.5% to 3.0% of glycerin and caprylyl glycol;
from 40.0% to 50.0% of water;
from 0.3% to 3.0% of sodium methyl stearoyl taurate, poloxamer 338 and stearic acid;
from 0.05% to 0.2% of a Citrus limon (lemon) fruit extract; and
from 0.01% to 0.2% of ascorbyl glucoside and tocopherol.

Wherein all percentages are based on the total weight of the tinted cosmetic sunscreen composition.

In an additional embodiment, the tinted cosmetic sunscreen composition of the present invention is use if for manufacturing a tinted product for the photoprotection of keratinous materials with high SPF and UVA/UVB protection, and with medium to high color coverage.

### EXAMPLES

### Examples 1 to 4

A suitable composition according to the state-of-the-art is as Examples 1 to 2 and a suitable tinted cosmetic sunscreen composition according to the present invention is as Examples 3 to 4, as follows:

TABLE 1
compositions from the state-of-the art and inventive compositions

| | | Ex. | Ex. | Ex. | Ex. |
|---|---|---|---|---|---|
| Function | Ingredient | 1 | 2 | 3 | 4 |
| pH | triethanolamine | 0.1 | 0.1 | 0.1 | 0.1 |
| adjuster | | | | | |
| Pigment | iron oxides | 1.3 | 6.1 | 6.1 | 1.3 |
| | titanium dioxide | 4.8 | — | — | 4.8 |
| Surfactant | Glyceryl stearate (and) | 1.3 | 1.3 | 1.8 | 1.8 |
| | peg-100 stearate | | | | |
| | Potassium cetyl phosphate | 1.3 | 1.3 | 1.5 | 1.5 |
| Fatty | isononyl isononanoate | 7.0 | 7.0 | 7.0 | 7.0 |
| compound | stearyl alcohol | 0.4 | 0.4 | 0.4 | 0.4 |
| | Copernicia cerifera | 2.0 | 2.0 | 2.0 | 2.0 |
| | (carnauba) wax | | | | |
| Filler | silica | 4.7 | 4.7 | 4.7 | 4.7 |
| | silica silylate | | | | |
| | aluminum starch | | | | |
| | octenylsuccinate | | | | |
| Fragrance | fragrance | 0.6 | 0.6 | 0.6 | 0.6 |
| polymer | ammonium | 0.6 | 0.6 | 0.6 | 0.6 |
| | acryloyldimethyltaurate/vp | | | | |
| | copolymer | | | | |
| | acrylates/C10-30 alkyl | | | | |
| | acrylate crosspolymer | | | | |
| preser- | phenoxyethanol | 0.9 | 0.9 | 0.9 | 0.9 |
| vative | chlorphenesin | 0.3 | 0.3 | 0.3 | 0.3 |
| | disodium EDTA | 0.1 | 0.1 | 0.1 | 0.1 |
| solvent | caprylyl glycol | 0.6 | 0.6 | 0.6 | 0.6 |
| | water | Q.S. | Q.S. | Q.S. | Q.S. |
| | glycerin | 2.0 | 2.0 | 2.0 | 2.0 |
| UV filter | Ethylhexyl triazone | 20.5 | 20.5 | 20.5 | 20.5 |
| | Titanium dioxide | | | | |
| | Octocrylene | | | | |
| | Diethylamino hydroxybenzoyl | | | | |
| | hexyl benzoate | | | | |
| | Ethylhexyl salicylate | | | | |
| | Butyl | | | | |
| | methoxydibenzoylmethane | | | | |
| Additional | Sodium methyl stearoyl taurate | 0.3 | 0.3 | 0.3 | 0.3 |
| Surfactant | Poloxamer 338 | 0.5 | 0.5 | 0.5 | 0.5 |
| | Stearic acid | 1.5 | 1.5 | 1.5 | 1.5 |
| Vegetal | Citrus limon (lemon) fruit | 0.1 | 0.1 | 0.1 | 0.1 |
| extract | extract | | | | |
| Vitamin | Ascorbyl glucoside | 0.11 | 0.11 | 0.11 | 0.11 |
| | Tocopherol | | | | |

### Example 5

### Stability Test

A stability test was performed in order to evaluate the stability of the tinted cosmetic sunscreen composition of Ex. 3 of the present invention, compared to the stability of a state-of-the-art composition as Ex. 2.

The compositions were placed in stability from 16 to 72 hours, after manufacture, in both containing glasses, which were then submitted to:
1 week at 55° C.;
1 month at 45° C., and
2 months at 45° C.

After these periods, the compositions were then microscopic and macroscopic evaluated, in order to assess their behavior of stability and physical chemistry.

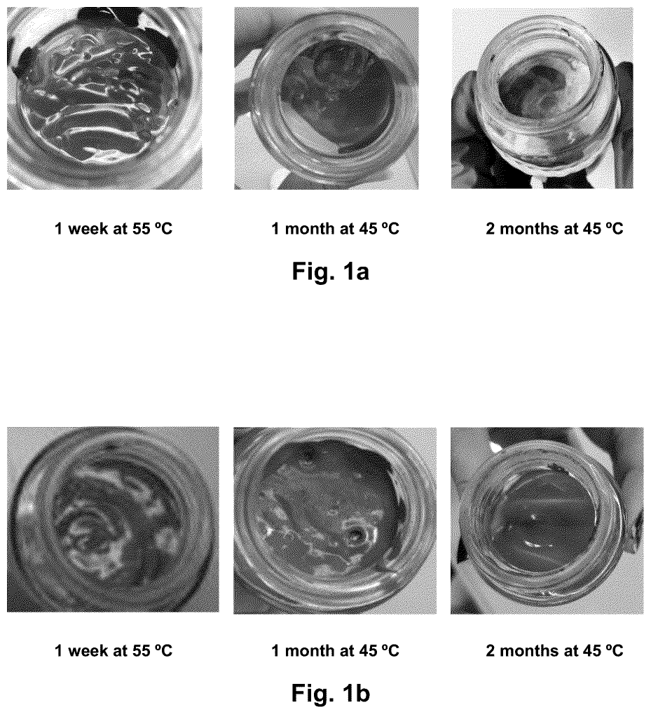

The results are demonstrated in FIG. 1, wherein FIG. 1a is related to the results of the state-of-the-art composition of Ex. 2 and FIG. 1b is related to the results of the inventive composition according to Ex. 3.

As can be seen, the composition according to Ex. 3 did not present instability of the formulation, nor migration of the pigments to the surface, whereas the composition of Ex. 2 was clearly instable, with a behavior of white migration spots in the surface.

1. A tinted cosmetic sunscreen composition, comprising:
(a) from 5.0 to 6.5% by weight, including all ranges and sub-ranges there between, based on the total weight of the composition, of at least one colored pigment selected from titanium dioxide, iron oxides and mixtures thereof;
(b) from 1.7 to 1.9% by weight, including all ranges and sub-ranges there between, based on the total weight of the composition, of glyceryl stearate (and) PEG-100 stearate;
(c) from 1.4 to 1.6% by weight, including all ranges and sub-ranges there between, based on the total weight of the composition, of potassium cetyl phosphate, and
(d) from 15.0 to 25.0% by weight, including all ranges and sub-ranges there between, based on the total weight of the composition, of at least one UV filter.

2. The composition, according to claim 1, wherein the at least one colored pigment ranges from 6.0 to 6.5% by weight, including all ranges and sub-ranges there between, based on the total weight of the composition.

3. The composition, according to claim 1, wherein the at least one UV filter is selected from octocrylene, diethylamino hydroxybenzoyl hexyl benzoate, ethylhexyl salicylate, ethylhexyl triazone, butyl methoxydibenzoylmethane, titanium dioxide, and mixtures thereof.

4. The composition, according to the claim 1, wherein the amount of the at least one UV filter ranges from 18.0 to 21.0% by weight, including all ranges and sub-ranges there between, based on the total weight of the composition.

5. The composition, according to the claim 1, further comprising Copernicia cerifera (carnauba) wax, ranging from 1.0 to 3.0% by weight, including all ranges and sub-ranges there between, based on the total weight of the composition.

6. The composition, according to claim 1, wherein it further comprises cosmetically acceptable ingredients selected from additional UV filters, active compounds, fatty compounds, fillers, perfume/fragrance, polymers, preserving agents, solvents, additional surfactants, vitamins, pH adjuster, silicones and mixtures thereof.

7. The composition, according to claim 1, wherein it presents a Sun Protection Factor (SPF) ranging of 50 to 60.

8. A use of a tinted cosmetic sunscreen composition, comprising:
(a) from 5.0 to 6.5% by weight, including all ranges and sub-ranges there between, based on the total weight of the composition, of at least one colored pigment selected from titanium dioxide, iron oxides and mixtures thereof;
(b) from 1.7 to 1.9% by weight, including all ranges and sub-ranges there between, based on the total weight of the composition, of glyceryl stearate (and) PEG-100 stearate;
(c) from 1.4 to 1.6% by weight, including all ranges and sub-ranges there between, based on the total weight of the composition, of potassium cetyl phosphate, and
(d) from 15.0 to 25.0% by weight, including all ranges and sub-ranges there between, based on the total weight of the composition, of at least one UV filter;
wherein said use if for manufacturing a tinted product for the photoprotection of keratinous materials with high SPF and UVA/UVB protection, and with medium to high color coverage.

9. A process for manufacturing a tinted cosmetic sunscreen composition, comprising:
(a) from 5.0 to 6.5% by weight, including all ranges and sub-ranges there between, based on the total weight of the composition, of at least one colored pigment selected from titanium dioxide, iron oxides and mixtures thereof;
(b) from 1.7 to 1.9% by weight, including all ranges and sub-ranges there between, based on the total weight of the composition, of glyceryl stearate (and) PEG-100 stearate;
(c) from 1.4 to 1.6% by weight, including all ranges and sub-ranges there between, based on the total weight of the composition, of potassium cetyl phosphate, and
(d) from 15.0 to 25.0% by weight, including all ranges and sub-ranges there between, based on the total weight of the composition, of at least one UV filter;
wherein the process comprises the steps of:
Step (A): Main Vessel—hot oily phase comprising oil raw materials in a temperature ranging from 75° C. to 80° C.;
Step (B): Side Vessel—hot aqueous phase comprising oil raw materials in a temperature ranging from 75° C. to 80° C.;
Step (C): the aqueous phase of step (B) is added to the oily phase of step (A), followed by mixing the mixture;
Step (D): the polymers are added to the mixture obtained in step (C), subsequently followed by a neutralization;
Step (E): the fillers are gradually added to the mixture obtained in step (D) and mixed until homogeneity, at a temperature of below 40° C.
